(19) United States
(12) Patent Application Publication
(10) Pub. No.: US 2022/0008620 A1
(43) Pub. Date: Jan. 13, 2022

(54) BONE SIALOPROTEIN FUNCTIONALIZED MATERIALS FOR DIRECTED BONE REGENERATION

(71) Applicants: Immundiagnostik AG, Bensheim (DE); Johannes Gutenberg-Universitat Mainz, Mainz (DE)

(72) Inventors: Franz Paul ARMBRUSTER, Bensheim (DE); Pol Maria ROMMENS, Mainz (DE); Ulrike RITZ, Ingelheim (DE)

(73) Assignees: Immundiagnostik AG, Bensheim (DE); Johannes Gutenberg-Universitat Mainz, Mainz (DE)

(21) Appl. No.: 17/291,767

(22) PCT Filed: Nov. 8, 2019
(86) PCT No.: PCT/EP2019/080768

§ 371 Date: May 6, 2021

(30) Foreign Application Priority Data
Nov. 8, 2018 (DE) 10 2018 128 017.3
Sep. 9, 2019 (DE) 10 2019 124 159.6

(51) Int. Cl. A61L 27/48; A61L 27/26; A61L 27/22; A61L 27/56; B33Y 80/00; B33Y 70/00; B29C 64/106

(52) U.S. Cl. CPC A61L 27/48; A61L 27/26; A61L 27/227; A61L 27/56; B33Y 80/00; B29K 2067/046; B29C 64/106; A61L 2300/412; A61L 2300/252; A61L 2430/02; B33Y 70/00

(57) ABSTRACT

A prosthetic polylactide or collagen-containing scaffold material for treating osseous defects and neogenesis of bone, obtained by printing a scaffold composed of strings of polylactide and porous microstructures which allow passage and ingrowth of bone tissue. A soluble mixture of BSP and/or collagen is provided, and BSP and/or collagen is applied onto the strings and in the pores of the printed body to obtain a prosthetic material which induces tissue-directed ingrowth of bone tissue as well as repair and healing of damaged or diseased bone tissues and lesions. The prosthetic material is osseo-inductive and osseo-conductive. The BSP in the prosthetic scaffold material induces a tissue-directed growth of osseous tissue. No undirectional callous or overgrowing bone and cartilage tissue is observed.

### FIELD OF THE INVENTION

The invention relates to 3D-printed composites comprising a resorbable polymer or copolymer, generally to gels and biocomposites made of base material comprising a resorbable polymer or copolymer, and in particular to methods and compositions for accelerating and improving the healing of bone and soft tissue lesions.

### BACKGROUND OF THE INVENTION

The stability of bone implants depends greatly on their ingrowth properties. For example, for dental implants to work, there must be sufficient bone in the jaw, and the bone has to be strong enough to hold and support the implant. Where there is insufficient or inadequate maxillary or mandibular bone in terms of depth or thickness, grafts are used in prosthetic dentistry to provide secure integration with the dental implant Conventional grafts include the patient's own bone (autografts), processed bone from cadaver (allografts), bovine bone or coral (xenografts), and synthetic bone-like or bone-mimetic materials.

The bone morphogenic proteins (BMP) are the only growth factors known to induce bone formation heterotopically. Supplementary doses of BMP boost the bone healing by inducing undifferentiated mesenchymal cells to differentiate into osteoblasts. Bone grafts and implants however shall result in a livevascular bone which is very much like natural U.S. Pat. No. 5,478,237 (Ishikawa) discloses an implant coated with a layer of hydroxyapatite, WO 021078759 (Stratec Medical AG) an implant having a layer of a porous metal oxide comprising amorphous and nanocrystallme calcium phosphate and hydroxyapatite, WO 02/085250 (KERWMED GrnbH) an implant wherein a coating of resorbable calcium phosphate phases contains adhesion and signal proteins such as bone sialoprotein (BSP), bone morphogenic protein (BMP), fibronectin, osteopontin (OPN), ICAM-I, VCAM and derivatives thereof. Further grafts and implants of this type are described in EP 1 166 804 A2 (Merck, Darmstadt) and WO 99/08730 (Children's Medical Center Corporation) DE 100 37 850 A1 (Jenissen H) and WO 03/059407 A1 (Straumann Holding AG) describe grafts and Implants treated with ubiqultin or transforming growth factor (TGF) or systemic hormones such as osteostatin, osteogenie and osteogrowth peptide (OGP), U.S. Pat. No. 7,229,545 82 (Biomet Deutschland GrnbH) teaches bone-analogous coatings made of a collagen matrix mineralised with calcium phosphate, EP 1 442 755 A1 (Depuy Producls) a bioactive ceramic coaling comprising osteogenic proteins OP-1, BMP-7 and non-collagenous bone matrix proteins, Osteogenic activities have further been reported for fibroblast growth factor (FGF), transforming growth factor-a (TGF-a), platelet-derived growth factor (PDGF), insulin growth factor (IGF) and family members of the foregoing. Zhang Q. et al describe in Biomacromolecules 2014, 15:84-94 the incorporation of recombinant human bone morphogenetic protein-2 (rhBMP-2). In an admixture with gelatin into a scaffold for a delivery of rhBMP-2 to stimulate improved bone regeneration via the supported growth and osteogenesis of human mesenchymal stem cells (hMSCs),

The prior art notwithstanding represents a problem as some implants often give rise to, for example, a condition called implantitis caused by an infection introduced during surgery. While irmplantitis can be dealt with by a course of antibiotics in the days prior and past surgery, a pre-emptive treatrment of peri-implantitis as well as improved wound and soft tissue healing would be more desirable. Aseptic loosening, inflammation reactions and long-term stability of endosseous implants further continue to remain a problem Despile bioactive coatings there is still a considerable period of lime between surgery and osteointegration until when bone grafts and implants can withstand typical pressure, shear and tensile forces. Moreover, uncontrolled and undirectional growth of the callous also give rise to dysfunctional bone tissues,

The aforementioned bone replacement materials are usually offered as a pasty filling material or coated metallic implants. However, reconstruction surgery also requires dimensionally stable bone replacement bodies for the immediate repair of bone-defects as well as dimensionally stable bone replacement parts, which continue to harden after setting and which are absorbed in the course of healing. In this connection, the healing involves and ingrowth of bone tissue. So it is sought, a bone replacement body or a hardening bone substitute material that is absorbed and promote bone healing, bone remodeling and bone growth. The prior art represents the problem.

### SUMMARY OF THE INVENTION

As a solution, the disclosure provides a polylactide prostetic body for treating osseous defects and neogenesis of bone, obtained by the steps of printing a scaffold composed of strings of polylactide and/or a gellous mixture of BSP and collagen, and adsorbing BSP and/or collagen onto the strings and in the pores of the microstructures. A polylactide scaffold is thereby obtained which induces tissue-directed ingrowth of bone tissue and repair and healing of damaged or diseased bone tissues and lesions. Further advantages and embodiments are described in the examples and the dependent claims.

In one aspect, the disclosure relates to a body for obtaining neogenesis of bone. The BSP may be recombinant human BSP.

In another aspect, the disclosure relates to a printed prosthesis for treating osseous defects and neogenesis of bone, wherein BSP is present in said mixture in a concentration from 1 μg BSP mL to 100 μg/mL, preferably from 2 μg rnL to 50 μg mL, more preferably from 3 μg/mL to 20 μg/m.

The BSP may be contained as a BSP-collagen material wherein said admixed collagen solution comprises fibrillar collagen selected from the group of collagen Type I, II, III, V, or XI.

In another aspect the disclosure relates to a material for treating osseous defects and neogenesis of bone, wherein said collagen solution comprises non-fibrillar collagen.

The disclosure also comprises a prosthesis for treating osseous defects and neogenesis of bone wherein said collagen solution is a gel solution comprising hydrolyzed collagen.

In another aspect, the disclosure relates to a polylactide prosthesis further comprising a gellous material, wherein said BSP-collagen solution comprises from 0.1 % (w/v) to 50% (w/v) collagen, preferably 0.5% (w/v) to 20% (w/v) collagen, more preferred from 1% (w/v) to 10% (w/v) collagen.

In one aspect, the disclosure relates to a polylactide prosthesis for treating osseous defects and neogenesis of bone which further comprises adsorbed natural or synthetic proteins which proteins are not originated from the complementary system.

In another aspect, the disclosure relates to a polylactide prosthesis for treating osseous defects and neogenesis of bone, which prosthesis comprises poly(Lys), poly(Glv-Pro-Hyp), tropocollagen and/or gelatine.

In one aspect, the disclosure relates to a polylactide prosthesis for treating osseous defects and neogenesis of bone, characterized in that its body has a total porosity from 10 to 90%, preferably from 20 to 70%, more preferred from 30 to 50%.

In another aspect, the disclosure relates to a use of the 3D-printed polylactide body as prosthesis in treating osseous defects and neogenesis of bone in the reconstructive surgery.

In one aspect, the disclosure relates to a method of making a prosthesis having neo-osseogenic properties comprising the steps of providing a solution with a physiologically effective amount of human BSP, and mixing said solution with collagen.

The present disclosure offers the advantage that BSP incorporation in collagen gels or polylactide is easily feasible and results in a continuous protein release. BSP bio-functionalised collagen gels and polylactide bodies have a cell growth stimulating effect beneficial for tissue regeneration, while offering a safe, non-tumorigenic environment. At lower BSP concentrations_{;} the gels enhance cell viability and sprout formation At higher BSP concentrations, osteoblast gene expression is enhanced. Notably, a concentration dependent effect can be observed in vivo already after three weeks from surgery,

The disclosure further provides a material for treating osseous defects and neogenesis of bone obtained by the steps of providing a material comprising collagen or polylactid or both, providing a physiologically effective amount of active human BSP, and combining said collagen and/or polylactide material and said physiologically effective amount of active human BSP before or during placement to obtain a material that furthers osseous repair and the healing of damage or diseased tissues and lesions. Advantages and embodiments of this disclosure are described in the examples and the dependent claims,

### BRIEF DESCRIPTION OF THE DRAWINGS

The present invention, its features and advantages will now be described by way of example only with reference to the accompanying drawings, wherein:

FIG. 1 shows a schematic representation of a process for evaluating the properties of collagen gels comprising a physiologically effective concentration of BSP;.

FIG. 2 is a diagram showing BSP release kinetics in collagen gels;

FIG. 3 is a schematic representation of the manufacturing steps of a 3-dimensional printed PLA replacement body which strings form pores of about 200 to 300 micrometer;

FIG. 4 is a bar diagramm showing the release cif BSP when coated with 1 or 10 μg BSP mixed with bovine collagen;

FIG. 5 shows microscopic analyses of various cell lines when grown on a 3D-printed polylactide polymer modified by collagen. BSP and vitronectin;

FIG. 6 shows microscopic analyses of osteosarcoma cells (SaOS-2) when grown on a 3D-printed polylactide body as described in FIG. 3 comprising BSP or not;

FIG. 7 shows microscopic analyses of endothelial cells (HUVEC) when grown on a 3D printed polylactide body as described in FIG. 3 comprising BSP or not;

FIG. 8 shows microscopic analyses of a co-culture of osteosarcoma cells (SaOS-2) and endothelial cells (HUVEC) when grown on a 3D printed body containing BSP or not

FIG. 9 shows the steps of implanting a 3D-printed polylactide body for regeneration of a portion of the rat femur;

FIG. 10 shows photos of crucial steps of the implant surgery and the removal of the implant after four and eight weeks of service;

FIG. 11 are x-ray representations of implanted 3D-printed polylactide replacement bodies containing collagen and/or BMP-7 after surgery and 2, 4, 6, and 8 weeks of service:

FIG. 12 are x-ray representations of implanted 3D-printed polylactide replacement bodies containing low and high concentrations of BSP after surgery and 2, 4, 6, and 8 weeks of service;

FIG. 13 is schematic representation of the steps for evaluating in vivo the relevant parameters when using BSP collagen gels in the calvarial bone defect model;

FIG. 13A displays images showing the bone regeneration in the rat calotte three weeks after surgery when using BSP collagen gels;

FIG. 13B displays images showing the bone regeneration in the rat calotte 8 weeks after surgery when using BSP collagen gels;

FIG. 14 shows a bar diagram of the results of a quantitative examination of the effects of a BSP collagen gel in the treatment of the calvarial bone defect model 3 weeks after surgery;

FIG. 15A shows hematoxylin-eosin stained histological sections of a treated bone defect using a BSP collagen gel three weeks after surgery;

FIG. 15B shows hematoxylin-eosin stained histological sections of a treated bone defect using a BSP collagen but eight weeks after surgery.

### DETAILED DESCRIPTION OF THE INVENTION

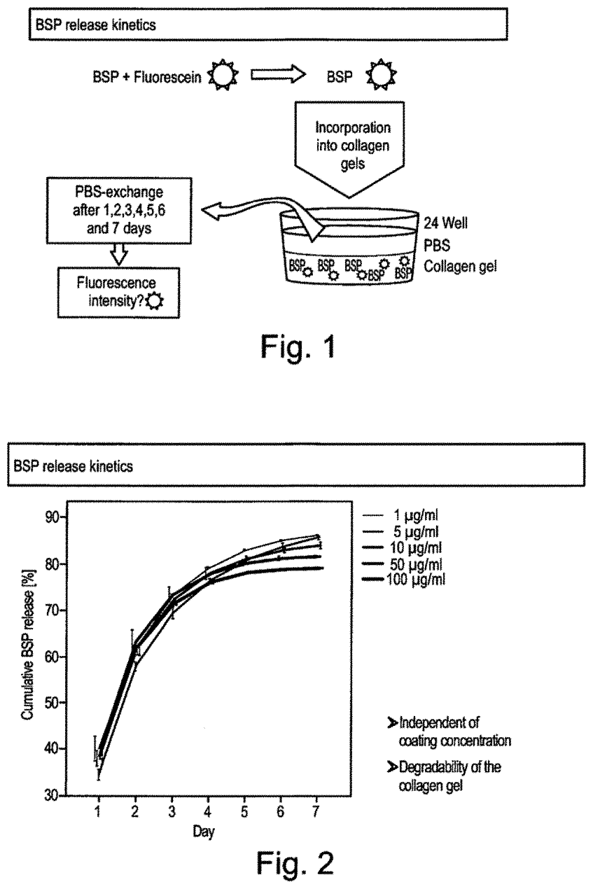

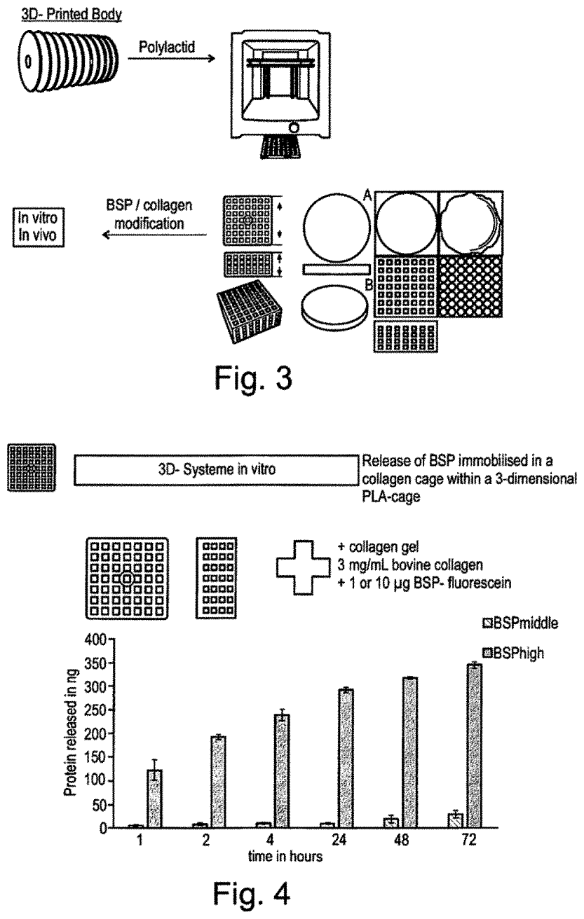

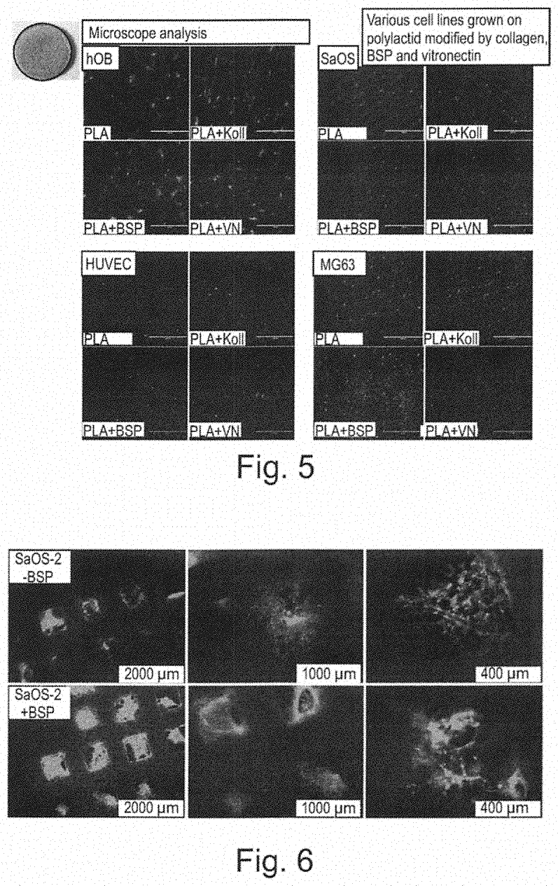

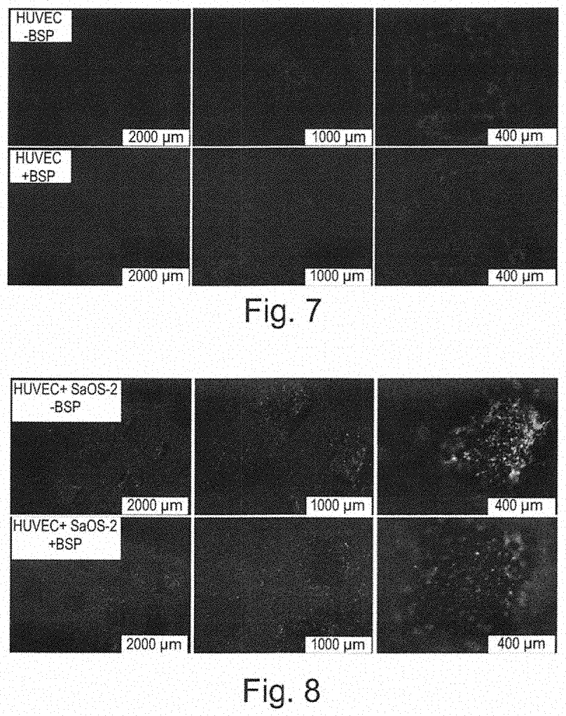

As a solution; the disclosure provides a prosthesis made of printed polylactide strings. The pores of the prosthesis have diameters of roughly 200 to 400 μm and therefore allow the entry of osteoblasts. The pores of the prosthesis may be filled and/or the strings be coated with a proteinaceous solution or gel comprising BSP and as a notable cofactor and stabilising agent collagen. The prosthesis for treating osseous defects and neogenesis of bone can be obtained by the steps shown in FIG. 3. The release of BSP and the degradability of the collagen gel or polylactide was examined and controlled as exemplified in FIGS. 1 and 2. Similar can be expected for degradable polylactide strings. The pores of the printed prosthesis may be filled either with a solution or a gel comprising BSP and collagen as exemplified in FIG. 4. In this way a physiological degradable body is provided as both the polylactide strings of the printed body as well as the collagen gel are degradable or absorbed in the course of the healing process. FIGS. 5-8 show various cell lines, notably human osteoblasts (hQB) and human osteosarcoma cells (SaOS-2), as well as HUVECs (human primary umbilical vein endothelial cells) grown in the presence and absence of BSP, vitronectin, collagen, PLA as indicated for comparative purposes. Generally, the presence of BSP lead to increased growth and differentiation of cells and notably hOBs.

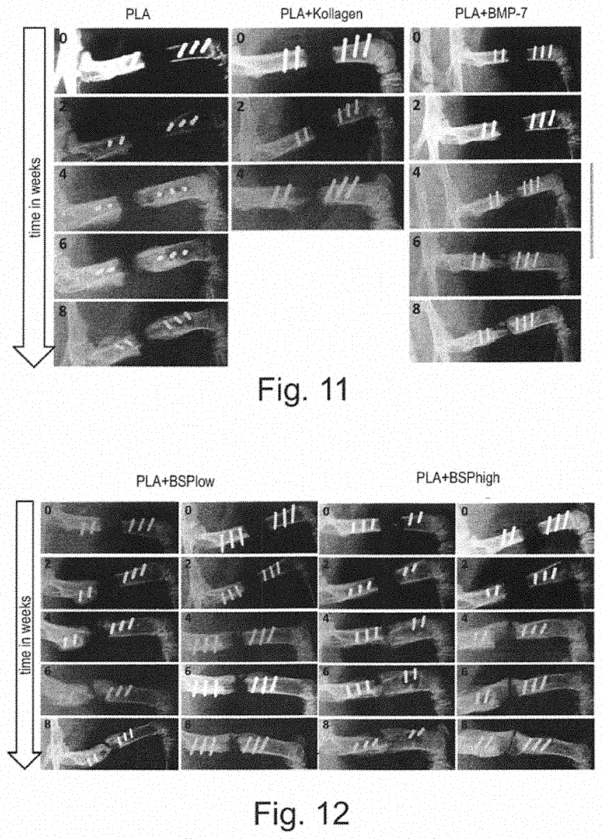

The enonmous advantage compared to the state-of-the-art employing bone morphogenic proteins (BMPs) is that BSP does not induce overgrowing of bone tissue and that the described combination of a printed polylactide body furthers directed or conducted neogenesis of bone material as exemplified in FIG. 12 FIG. 11 shows the 3D printed prosthesis but coated or trenched with collagen or bone morphogenic protein 7 (BMP-7). The bone morphogenetic proteins (BMPs) are growth factors and received their name by the discovery that they can induce the formation of bone and cartilage BMPs are now considered morphogenetic signals which orchestrate tissue architecture throughout the body.

The functioning of BMP signals in physiology is further emphasized by the multitude of roles for dysregulated BMP signalling in pathological processes. Recombinant human BMPs (rhBMPs) are meanwhile used in orthopedic applications and recombinant human BMP-2 and BMP-7 have received Food and Drug Administration (FDA)-approval for some uses. However, rhBMP-2 and rhBMP-7 can cause an overgrowing of bone.

While new additional bone tissue is formed by the signal action of BMP-7, the bone tissue is not formed in physiological or natural direction of the femur or calvarial defect. In other words, the newly formed bone tissue is not directional but callous-like. Even in the case of even a high concentration of BSP (see FIG. 12, eight weeks), we found that a prosthesis containing BSP—adsorbed or contained within a collagen gel or polylactide—does not only induce and support the formation of new bone tissue but that the BSP induces in this combination a tissue-directed formation of bone. It is therefore further obvious to print and place a 3D-printed body or prosthesis in a way that the pores are osseous conductive so that the cell and osseous-inductive signaling of the BSP can be fully used.

### EXAMPLE 1

### BSP Coated Printed Prosthesis in Rat Femur

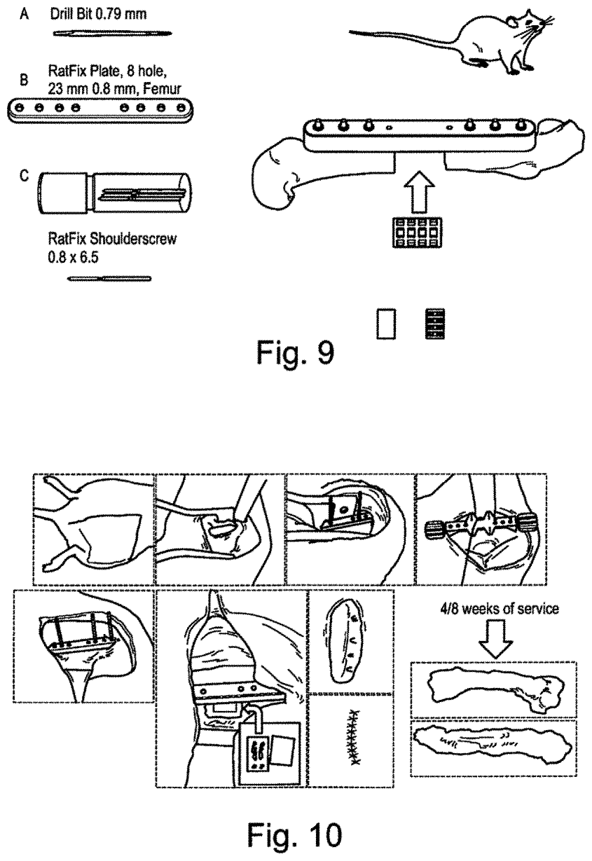

The animal experiment was approved by the competent Rhineland-Palatinate State Investigation Office (LUA). The steps of this animal experiment can be taken from FIGS. 9 and 10. In brief, a piece of the rat femur was cut out as indicated and repaired by a 3-D printed polylactide prosthesis which was put in place as indicated using an 8-hole fixing plate. FIG. 9 shows the principle and FIG. 10 the surgery steps. The rat femur was X-rayed immediately after surgery and after 2, 4, 6 and 8 weeks as shown in FIGS. 11 and 12. The fixing plate and the prosthesis are not sufficiently electron dense to be visible. The femurs were further histologically examined (results not shown).

The enormous advantage compared to the state-of-the-art (FIG. 11) employing bone morphogenic proteins (BMPs) or collagen is that BSP does not induce overgrowing of bone tissue and that the described combination of a printed polylactide body furthers directed or conducted neogenesis of bone material as exemplified in FIG. 12 (see in particular X-ray photos after eight weeks). FIG. 11 shows printed polylactide prosthesis coated or trenched with collagen or bone morphogenic protein 7 (BMP-7). The bone morphogenetic proteins (BMPs) induced formation of bone but the growth of bone was not directional. BMPs are generally considered morphogenetic signals which orchestrate tissue architecture throughout the body. The coated BMP however leads to dysregulated BMP signalling in pathological processes while recombinant human BMP-2 and BMP-7 have received Food and Drug Administration (FDA)-approval for some orthopedic applications.

However, rhBMP-2 and rhBMP-7 cause overgrowth and non-directional growth of bone. As shown in FIG. 11, while additional bone tissue is formed by the action of BMP-7, the additional bone tissue is not formed in the direction of the femur. Consequently, he newly formed bone tissue is non-directional but callous-like and therefore not serving or assisting the functions of the femur.

Referring to FIG. 12 (eight weeks), we could observed that a placed printed polylactide prosthesis with low or high concentration of BSP induced directional formation of new bone tissue. The prosthesis was a porous body containing BSP, adsorbed onto the polylactide strings. It is therefore obvious to print and place the prosthesis in a way that the pores are osseous conductive so that the cell and osseous-inductive signaling of the BSP is fully effective.

### EXAMPLE 2

### BSP Collagen Prosthesistreating in a Calvarial Bone Defect

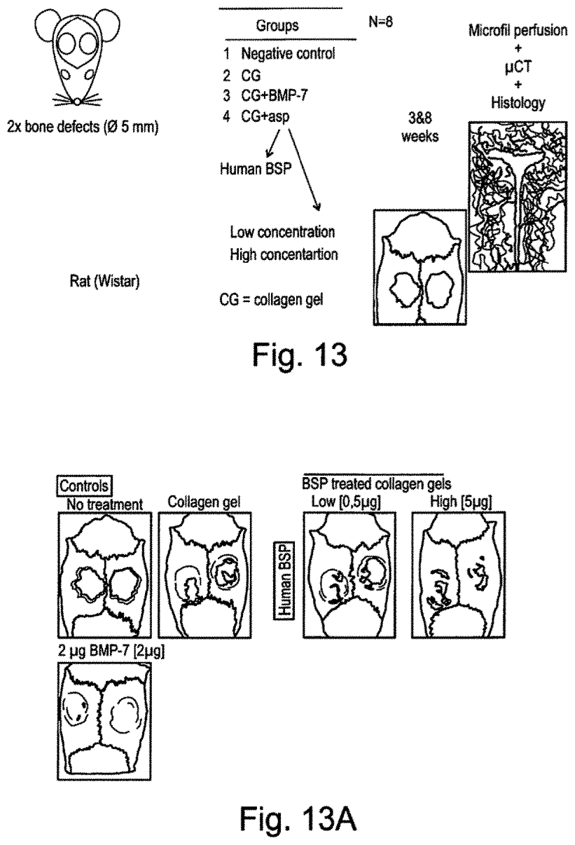

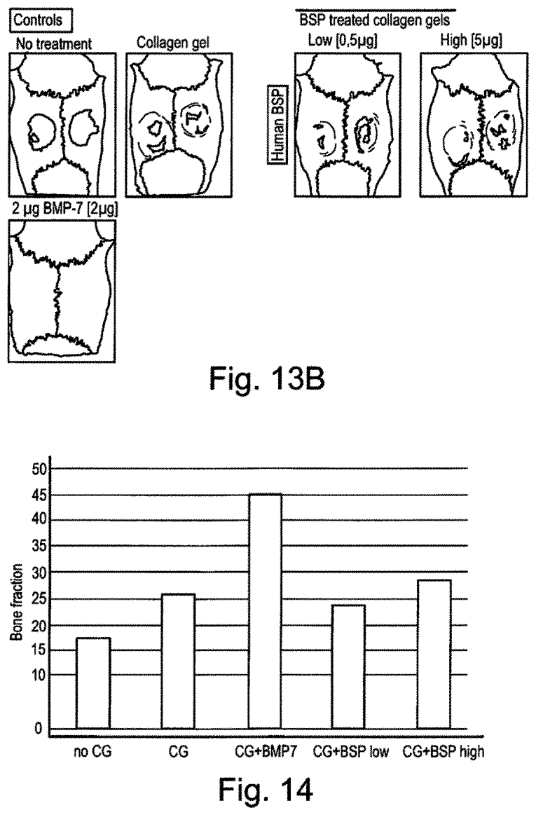

Referring to FIG. 13, the prosthesis was used to treat a calvarial bone defect. The animal experiment was approved by the competent Rhineland-Palatinate State investigation Office (LUA). The rats were operated under general anesthesia and placed in each case two boreholes with a diameter of 2.5 mm—a bone defect of critical size. Then, BSP collagen gels were placed into the borehole to cover it up. After an observation periods of 3 and 8 weeks, the animals were anesthetized and decapitated.

The skulls were fixed in formalin for one week and prepared for fJeT imaging. The animal experiment included following groups:

a) Negative control: i) untreated borehole defect, and ii) treated borehole defect with collagen only.

b) Positive control: treated borehole defect with BMP-7 (2 μg).

c) Experiment: i) BSP treated collagen gels with 0.5 μg, and ii) BSP treated collagen gels with 5 μg.

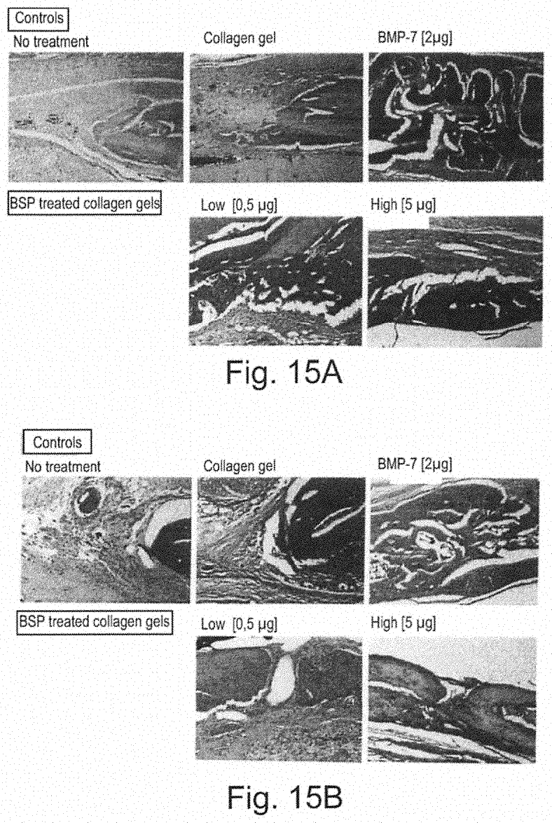

The BSP treated animals showed improved osseous regeneration compared to controls already three weeks after surgery. Notably, the growth of bone tissue was more homogenous and functional in the BSP treated animals, cf FIGS. 15A-B The additional bone growth in the positive control (BMP-7) was similar but with signs of osseous overgrowth.

### CONCLUSIONS

A prosthetic polylactide or collagen-containing scaffold material for treating osseous defects and neogenesis of bone, obtained by the steps of printing a scaffold composed of strings of polylactide and porous microstructures which allow passage and ingrowth of bone tissue. A soluble mixture of BSP and/or collagen is provided, and BSP and/or collagen is applied onto the strings and in the pores of the printed body to obtain a prosthetic material which induces tissue-directed ingrowth of bone tissue as well as repair and healing of damaged or diseased bone tissues and lesions The prosthetic material has thereby been made osseo-inductive and osseo-conductive. The BSP in the prosthetic scaffold material induces a tissue-directed growth of osseous tissue. No undirectional callous or overgrowing bone and cartilage tissue is observed.

1-10. (canceled)

11. Method of treating osseous defects and for neogenesis of bone material comprising the steps of:
providing an implant scaffold or prosthesis comprising biodegradable polylactide (PLA) or collagen which allows passage and ingrowth of osteoblasts and osteoclasts, providing a gel or liquid solution comprising collagen or polylactide,
providing a physiologically effective amount of active human BSP, and
combining said collagen or polylactide material with a physiologically effective amount of active human BSP to obtain a gel or liquid mixture and/or
treating and/or impregnating said implant scaffold or prosthesis with said gel or liquid mixture prior to surgical placement to obtain a scaffold material or prosthesis effective to release BSP to further tissue-directed repair and healing of damaged or diseased bone tissues and lesions.

12. The method of claim 11, wherein BSP is human recombinant BSP.

13. The method of claim 11, wherein said human BSP is present in the gel or liquid mixture in a concentration from 1 μg/mL to 100 μg/mL, preferably from 2 μg/mL to 50 μg/mL, more preferably from 3 μg/mL to 20 μg/mL.

14. The method of claim 11, wherein said gel or liquid mixture further comprises fibrillar collagen selected from the group of collagen Type I, II, III, V or XI.

15. The method of claim 11, wherein said gel or liquid mixture comprises non-fibrillar collagen.

16. The method of claim 11, wherein said gel or liquid mixture comprises hydrolyzed collagen.

17. The method of claim 11, wherein said scaffold or prosthesis is 3D-printed and said gel or liquid mixture with a physiologically active amount of BSP comprises 0.1% (w/v) to 50% (w/v) collagen, preferably 0.5% (w/v) to 20% (w/v) collagen, more preferred from 1% (w/v) to 10% (w/v) collagen.

18. The method of claim 11, wherein the scaffold or prosthesis is 3D-printed and said gel or liquid mixture with a physiologically active amount of BSP further comprises at least one member selected from the group consisting of poly(Lys), poly(Gly-Pro-Hyp), tropocollagen and gelatin.

19. The method of claim 11, wherein the scaffold or prosthesis is 3D-printed with a total porosity from 10 to 90%, preferably from 20 to 70%, more preferred from 30 to 50%.

20. The method of claim 11, wherein the scaffold or prosthesis is printed for conducted or tissue-directed neogenesis of a homologous bone material.

21. The method of claim 11, wherein the prosthetic scaffold material has been made osseous inductive and osseous-conductive without causing non-directional callous or overgrowing of bone material or cartilage tissue.

22. Method of treating osseous defects and lesions comprising the steps of:
3D-printing a prosthetic implant scaffold which allows passage and ingrowth of osteoblasts and osteoclasts, providing a gel-like or liquid solution comprising collagen or polylactide,
providing a physiologically effective amount of active human BSP, and
combining said collagen or polylactide material with a physiologically effective amount of active BSP to obtain a gel or liquid mixture and/or
treating and/or impregnating said prosthetic implant scaffold with said gel or liquid mixture prior surgical placement to obtain a prosthetic implant which is osseous inductive and osseous-conductive without causing non-directional callous or overgrowing of bone material or cartilage tissue.
